(19) United States
(12) Patent Application Publication
(10) Pub. No.: US 2022/0241065 A1
(43) Pub. Date: Aug. 4, 2022

(54) CLAMPING ELEMENT AND SETTING ASSEMBLY

(71) Applicant: H & B ELECTRONIC GMBH & CO. KG, Deckenpfronn (DE)

(72) Inventors: Wilfried WEBER, Schopfloch (DE); Tobias MORLOK, Mötzingen (DE)

(73) Assignee: H & B ELECTRONIC GMBH & CO. KG, Deckenpfronn (DE)

(21) Appl. No.: 17/622,900

(22) PCT Filed: Jan. 26, 2021
(86) PCT No.: PCT/EP2021/051692

§ 371 Date: Dec. 27, 2021

(30) Foreign Application Priority Data
Feb. 28, 2020 (DE) 10 2020 105 381.9

(51) Int. Cl. A61F 2/08

(52) U.S. Cl. CPC A61F 2/0805; A61F 2002/0817; A61F 2220/0016; A61F 2002/0888; A61F 2002/0852

(57) ABSTRACT

A setting assembly for a clamped fixation of a tissue strand, such as a tendon or a band in particular, to a securing recess, which is recessed into a bone, has a clamping element which has a clamping section that tapers along a central axis of the clamping element towards a first end, a recess that is provided on a second end facing away from the first end for passing through the tissue strand, and a tool receiving area that is recessed onto the first end and has a first contact for applying a force acting in a pushing direction and a second contact for applying a force acting in a pulling direction opposite the pushing direction. The setting assembly additionally has an applicator which has an insertion end for releasably connecting to the tool receiving area, the insertion end having a first mating contact which can be placed against the first contact and a second mating contact which can be placed against the second contact means. The tool receiving area has first and second rotation locks, and the insertion end of the applicator has a rotating device which can be placed against the first and second rotation locks in a first rotational direction and a second rotational direction opposite the first rotational direction in order to transmit first and second torques.

The invention relates to a setting assembly for a clamped fixation of a tissue strand, such as in particular a tendon or a band, to a securing recess let into a bone, according to the preamble of claim 1, as well as to a clamping element and an applicator for use in such a setting assembly. In this regard, the setting assembly comprises a clamping element having a clamping portion that tapers toward a first end along a central axis of the clamping member. At a second end remote from the first end, the clamping element further includes a recess for passing the tissue strand therethrough. Furthermore, the clamping element comprises a tool receptacle recessed at the first end. At this tool receptacle, first contact means are provided for a force application effective in a pushing direction and second contact means are provided for a force application effective in a pulling direction opposite to the pushing direction. In addition, the setting assembly has an applicator on which an insertion end is provided for releasable connection to the tool receptacle. In this connection, first mating contact means are provided at the insertion end, which mating contact means can be applied to the first contact means in the push direction for the application of force by the applicator which is effective in the push direction. In addition, second mating contact means are provided at the insertion end, which can be applied to the second contact means in the pulling direction for the application of force by the applicator in the pulling direction.

The second contact means advantageously have a fixing region for producing a form-fitting, quasi-form-fitting or force-fitting connection with the applicator, which connection acts in the pulling direction. As a result, the clamping element can be gripped behind by a correspondingly configured applicator or cooperate therewith in order to form a positive or non-positive connection which is effective in the pulling direction. In this way, relatively high tensile forces can be applied to the clamping element via the connection between the applicator and the clamping element in order to be able to position the latter precisely in the securing recess, for example when clamping the tissue strand to be fixed. Advantageously, the second contact means thereby have at least one fixed stop which is effective in the pulling direction, whereby relatively high pulling forces can be transmitted from the applicator to the clamping element during the setting operation and, in particular, for correcting the position of the clamping element at the end of the setting operation. Alternatively, or additionally thereto, the second contact means may comprise at least one force-locking element which is effective in the tensile direction, for example elastically deformable or displaceable, in order, for example, to limit the tensile forces which can be absorbed by the connection. This limitation of absorbable tensile forces can thereby serve, for example, to prevent damage to the clamping element or injury to the tissue strand to be fastened.

Such fixation devices are used in arthroscopic or open surgery to anchor tendon grafts, bands, or similar tissues in the bone, such as in cruciate band replacement. In the state of the art, various fixation systems are used for this purpose, such as various suture anchor systems, interference screws or staples, the Transfix® system, the Bio-Tenodese-Schraube®, the Endo-Button®, the EndoPearl®, the Sutur-Disc® and the like. Modern fixation systems for cruciate band surgery today have the requirement of fixation close to the joint. Furthermore, the healing of the graft should always take place predominantly in the area of the cortical bone in order to avoid negative graft movements within the bone canal.

From WO2011141067A1 a setting assembly is known, which serves to fix a tendon to a securing recess via a clamping element. The securing recess is formed as a blind hole with an additional undercut, so that the securing recess tapers at least in sections towards an entry hole. The clamping element is adapted in size to the securing recess such that it can be displaced into the securing recess via the entry hole with the aid of an applicator. In this position within the securing recess, a guide thread is then deflected as well as a chord attached to the end thereof, which is pulled by means of the guide thread into the securing recess, around the clamping element and out of the securing recess again with one end. The deflected chord, together with the clamping element, thereby produces a total cross-section which is larger than a free cross-section of the inlet opening. By pulling on the chord, the deflected chord together with the clamping element can thus be clamped in the securing recess.

A disadvantage of the known clamping element is that it can only be fixed in relation to the applicator to a limited extent. As a result, only a limited or relatively awkward correction of the position of the clamping element within the securing recess is possible during placement. In some cases, this can lead to the fact that an optimum clamping position relative to the securing recess cannot be achieved, in particular in the case of a tensile force already applied to the tissue strand during fastening. Furthermore, when threading and deflecting the tissue strand within the securing recess, canting often occurs with the known clamping elements.

It is the task of the invention to avoid the mentioned disadvantages in a generic setting assembly and to enable a simpler and more precise positioning as well as, if necessary, a subsequent position correction of the clamping element. In addition, it is the task of the invention to enable a trouble-free passage and deflection of the tissue strand to be fixed.

This task is solved by a setting assembly having the features of claim 1. In this case, the tool receptacle additionally has first rotation-locking means and second rotation-locking means, and the insertion end of the applicator has rotating means which can be applied to the first and second rotation-locking means in a first direction of rotation and in a second direction of rotation opposite to the first direction of rotation for the transmission of first and second torques. This allows the clamping element to be additionally rotated about the central axis in both rotational directions during the setting operation by the applicator, in addition to being displaced in the pushing and pulling directions. In this way, the forces and moments applied to the applicator during the setting process can be transmitted to the clamping element in all directions via the connection with the clamping element, which enables simple and precise positioning of the clamping element within the securing recess and subsequent correction even of clamping forces already acting on the clamping element.

In a particularly preferred embodiment, the clamping element and the applicator can be brought into a connection position in which the contact means are simultaneously in contact with both counter-contact means and the rotating means are simultaneously in contact with both rotation-locking means. In this respect, it is particularly preferred if the first and second rotation-locking means are formed by rotation stops acting in the direction of rotation. In this way, a positive connection can be produced between the clamping element and the applicator in both directions of rotation, whereby relatively high holding or displacing torques can be transmitted from the applicator to the clamping element in both directions of rotation. Through this, in the connection position, a very direct transmission of forces and moments from the applicator to the clamping element is possible in all directions and, in particular, without relative movement between the applicator and the clamping element, such as in the form of empty paths. This in turn enables a very stable and precise positioning of the clamping element and in particular also a subsequent correction of the clamping element within the securing recess.

In this context, it is also particularly advantageous if a positive connection is formed both between the contact means and counter-contact means and between the rotating means and the anti-rotating means, in order to be able to transmit even relatively large forces and torques to the clamping element by means of the applicator, in particular without relative movement between the applicator and the clamping element. In this way, in particular even when a relatively large tensile stress is applied to the strand of fabric to be fastened, an unwanted displacement or rotation of the clamping element during clamping to the recess can be prevented.

In this context, it is advantageous if the first mating contact means are formed by a distal shank end of an applicator shank and the second mating contact means are formed by latching means which are displaceable between a release position and a latching position and, in the latching position, project beyond a lateral surface of the applicator shank for the formation of a force-fitting or form-fitting engagement with the undercut recess of the tool receptacle. In this way, both a stable connection for setting the clamping element and a secure release of the applicator from the clamping element after its positioning in the desired clamping position are possible.

Advantageously, the latching means comprise an elastic spring ring, whereby the second mating contact means can be manufactured in a particularly simple and inexpensive manner.

Alternatively, or additionally thereto, the latching means comprise latching bodies which are displaceable and fixable in the latching position in order to be able to ensure a particularly stable and easily releasable connection between the applicator and the clamping element.

In this respect, it is advantageous if the applicator has an adjusting pin which is displaceable within the applicator shaft and which blocks a radial inward displacement of the latching bodies in a latching position and releases them in a release position, thereby enabling a simple and stable fixing of the latching bodies.

In a particularly preferred embodiment, the insertion end has rotating means which can be applied to the first rotation-locking means of the tool receptacle and interact with these in the first direction of rotation, and rotating means which can be applied to the second rotation-locking means and interact with these in the second direction of rotation, and which are formed by a polygonal profile formed at the distal end of the applicator shaft. This allows relatively high torques to be transmitted from the applicator to the clamping element.

Alternatively, or additionally thereto, the rotating means comprise a bayonet cam which is engageable with the bayonet receptacle of the tool receptacle. The formation of such a bayonet catch thereby enables a particularly convenient connection and disconnection of the applicator with respect to the clamping element. In addition, relatively large forces can be transmitted to the bayonet mount of the clamping element via the bayonet cam, both in the pushing direction and in the pulling direction.

In this case, it is particularly preferred if the bayonet receptacle has a first bayonet receptacle and a second bayonet receptacle arranged offset thereto in the direction of the central axis, and a first bayonet cam which can be accommodated in the first bayonet receptacle and a second bayonet cam which can be accommodated in the second bayonet receptacle and is held rotatably with respect to the first bayonet cam are provided on the applicator. In this way, by means of the bayonet catch, a direct positive connection between the applicator and the bayonet receptacle can be produced both in the pushing and pulling direction and in both directions of rotation about the central axis of the clamping element.

In this case, it is also advantageous if the first bayonet cam is connected to a first handle part via a first partial shaft and the second bayonet cam is connected to a second handle part of the applicator via a second partial shaft. In this way, both bayonet cams can be rotated relative to one another in order, for example, to be brought into a common alignment for establishing and releasing the connection with the clamping element and in order, for example, to be aligned perpendicularly to one another for establishing the rotation locks acting in both directions of rotation.

Furthermore, the above-mentioned task is solved by a clamping element suitable for use in a setting assembly in one of the above-mentioned embodiments. The clamping element has two lateral contact surfaces extending between the recess and the first end on two sides of the clamping section facing away from each other. The lateral contact surfaces thereby serve to contact the deflected tissue strand and to generate retaining clamping forces on the tissue strand.

In a particularly favorable embodiment, the recess has a closed eyelet shape, in particular an elliptical shape. This provides a circumferential and thus particularly stable connection of the clamping element with the tissue strand threaded therethrough, so that the latter is compulsorily carried along when the clamping element is displaced. Thus, a precise deflection of the tissue strand at the clamping element can be ensured.

In an alternative preferred embodiment, the recess is U-shaped and open towards the second end. Due to this open U-shape, a relatively large free cross-section of the recess can be made available, into which the tissue strand to be fastened can thus be threaded and passed through more easily. The open U-shape at the second end is thereby independent of the embodiment of the clamping wedge at the first end. In particular, the U-shaped recess at the second end of each clamping wedge can be formed independently of the embodiment of the tool receptacle at the first end or independently of whether such a tool receptacle is used at all.

Furthermore, it is advantageous if a guide groove is let into at least one of the lateral contact surfaces in the direction of the recess in order to facilitate threading and guiding the tissue strand through the recess of the clamping element.

In this regard, it is convenient if the guide groove has a groove bottom profile with different pitches to allow more comfortable displacement along the clamping element and easier deflection of the tissue strand to be fastened.

In addition, it is advantageous if the groove bottom profile forms a convex contour in the clamping section in order to generate a maximum deflection of the tissue strand in contact with the lateral contact surfaces and thus relatively large clamping forces during the clamping process. This ensures that the tissue strand is securely fixed to the securing recess.

Preferably, the guide groove opens into the recess via a rounded deflection area in order to avoid jamming as far as possible when threading into the recess.

Furthermore, it is convenient if an additional thread guide is let in at the recess, in which a guide thread connected to the tissue strand to be fastened can be accommodated. In this way, the guide thread can be fixed to the recess before the clamping element is inserted into the securing recess and can be guided along a predefined path during the threading process of the tissue strand in order to ensure, in turn, secure guidance of the subsequent tissue strand.

Advantageously, the additional thread guide thereby has an exit gap via which the guide thread can be displaced out of the thread guide in order to enable the passage of the tissue strand through the recess. In particular, the exit gap may have a width which is slightly smaller than a cross-section of the guide thread, so that the guide thread can only exit the thread guide when the exit gap is elastically widened, or the guide thread cross-section is elastically narrowed.

Furthermore, it is advantageous if the additional thread guide is designed as a double thread guide for two guide threads guided at a distance from each other. This enables a more stable guidance of the tissue strand, in which the tissue strand can also be slightly spaced apart from the clamping element and thus be displaced with an increased deflection radius. This allows it to be deflected more safely and in particular without jamming on the clamping element.

Furthermore, it is advantageous if roughenings, such as knob or tooth structures, are formed on the lateral contact surfaces for increased friction with respect to an adjacent tissue strand, in order to be able to clamp the tissue strand deflected at the clamping element in a particularly positionally stable manner at the securing recess.

In this case, it is advantageous if the roughenings have toothed structures and the teeth of these toothed structures are each at least partially inclined in the direction of a threading direction, in order to be able to ensure a particularly high frictional resistance to a displacement of the tissue strand against a threading direction.

In this case, the roughenings preferably extend at the height of the convex section of the groove bottom profile with respect to the central axis of the clamping element, whereby the roughenings can act in particular in the area with the highest clamping forces and thus generate a particularly high frictional resistance.

Further, the above problem is solved by an applicator for use in a setting assembly according to any of the above embodiments.

It is pointed out that all the features of the object according to the invention described above are interchangeable or combinable with one another, provided that an interchange or combination thereof is not precluded for technical reasons.

An exemplary embodiment of the invention is shown in the Figures. It shows:

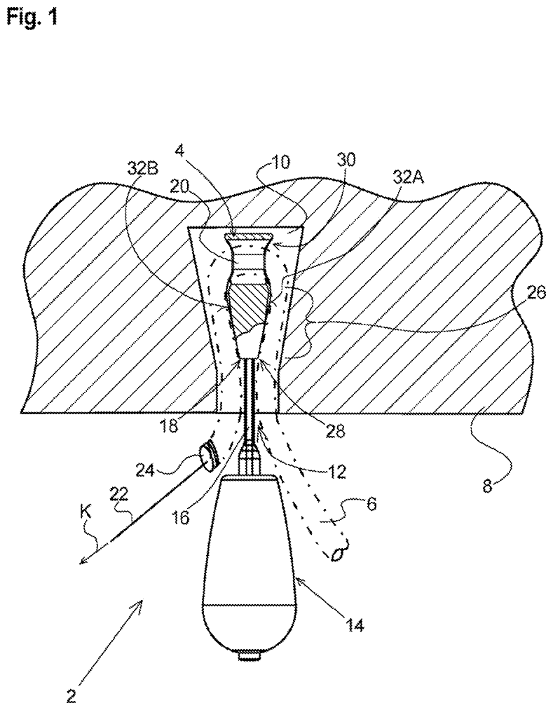

FIG. 1 a partial sectional view of a setting assembly during a setting operation,

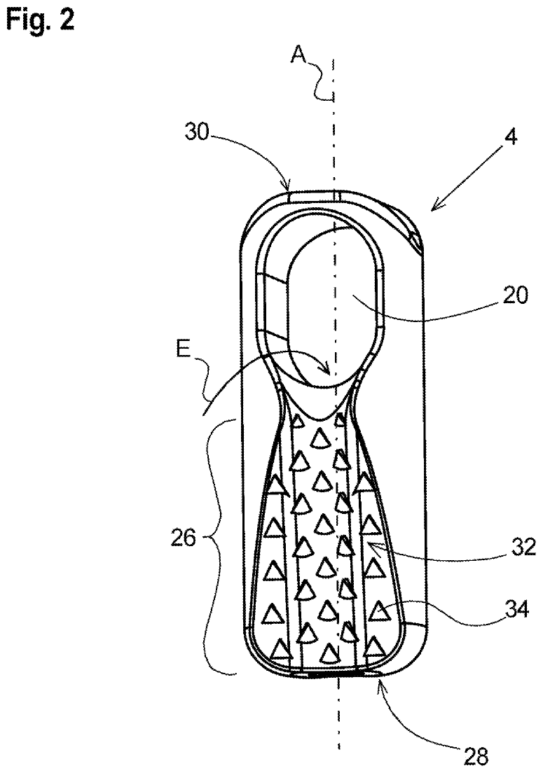

FIG. 2 a perspective view of a clamping element of the setting assembly according to FIG. 1,

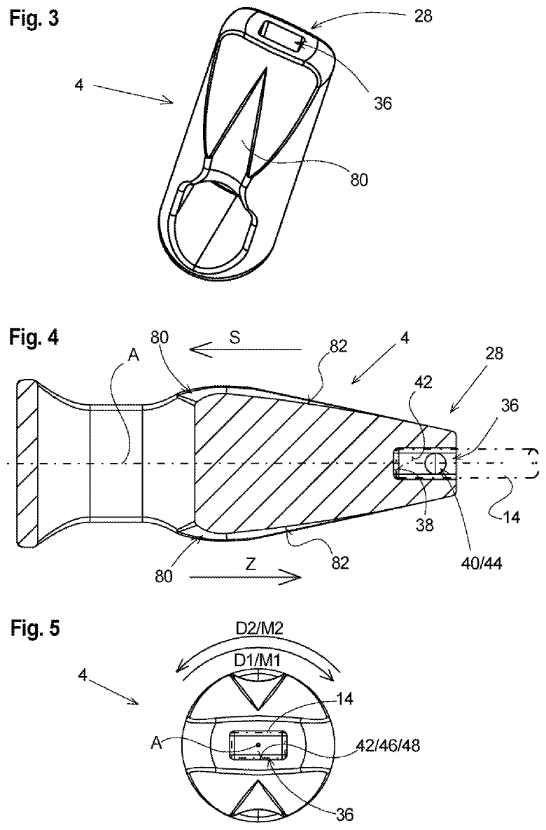

FIG. 3 a perspective view of an alternative embodiment of the clamping element with a rectangular tool receptacle,

FIG. 4 a longitudinal section through the clamping element according to FIG. 3,

FIG. 5 a top view of a first end of the clamping element according to FIG. 3,

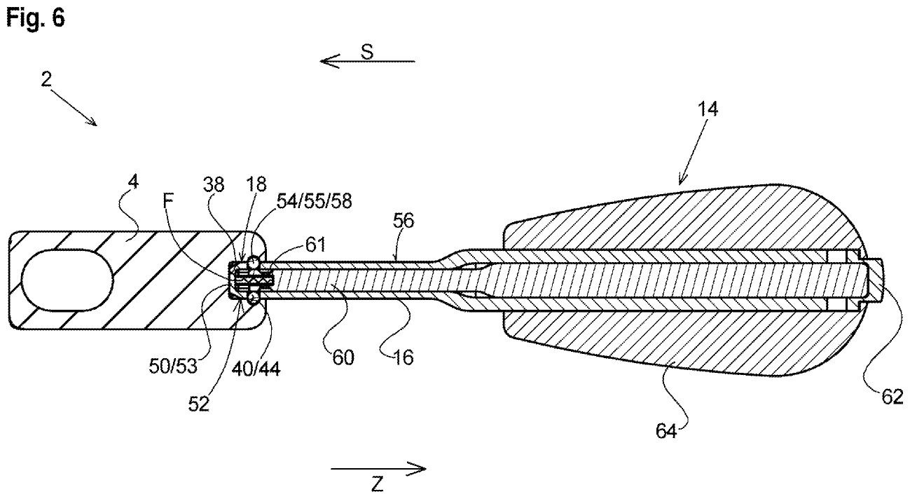

FIG. 6 a longitudinal section through the clamping element according to FIG. 3 in the attached state on an adapted applicator,

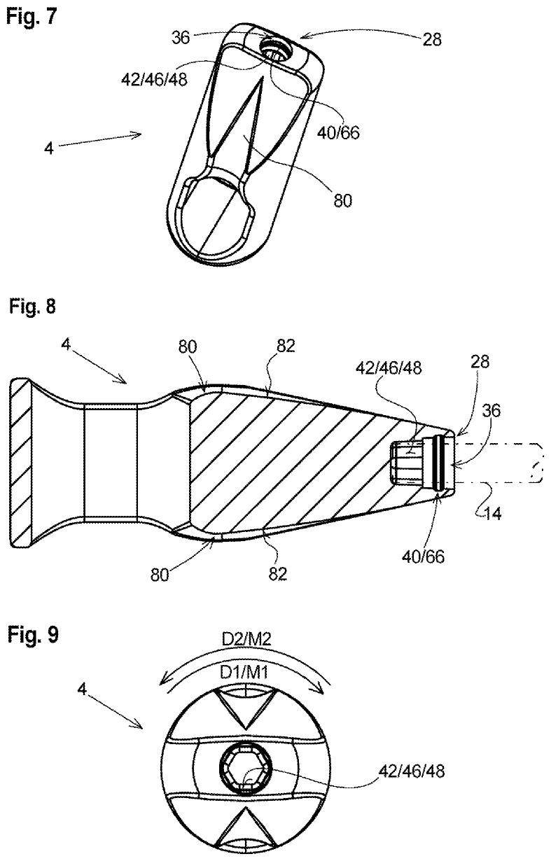

FIG. 7 a perspective view of a further alternative embodiment of the clamping element with a hexagonal tool receptacle,

FIG. 8 a longitudinal section through the clamping element according to FIG. 7,

FIG. 9 a top view of a first end of the clamping element according to FIG. 7,

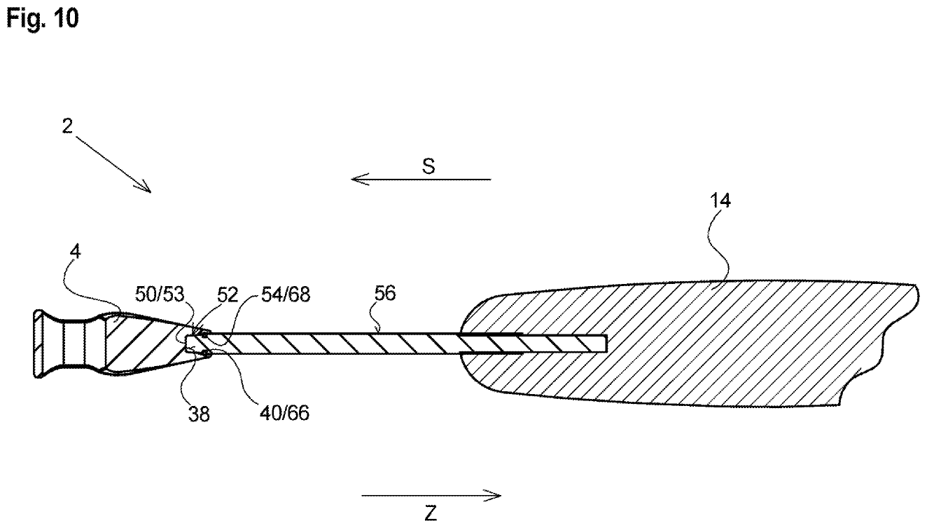

FIG. 10 a longitudinal section through the clamping element according to FIG. 7 in the attached state on an adapted applicator,

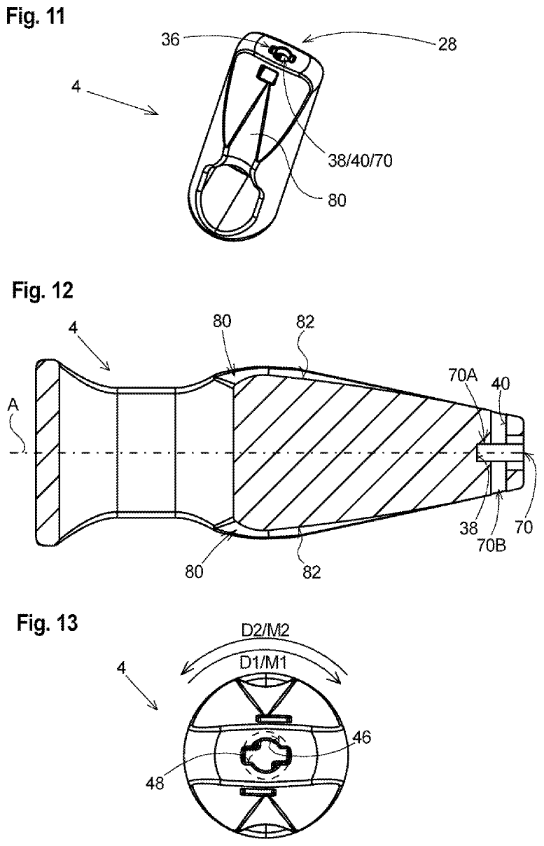

FIG. 11 a perspective view of a further alternative embodiment of the clamping element with a bayonet mount,

FIG. 12 a longitudinal section through the clamping element according to FIG. 11,

FIG. 13 is a top view of a first end of the clamping member shown in FIG. 11,

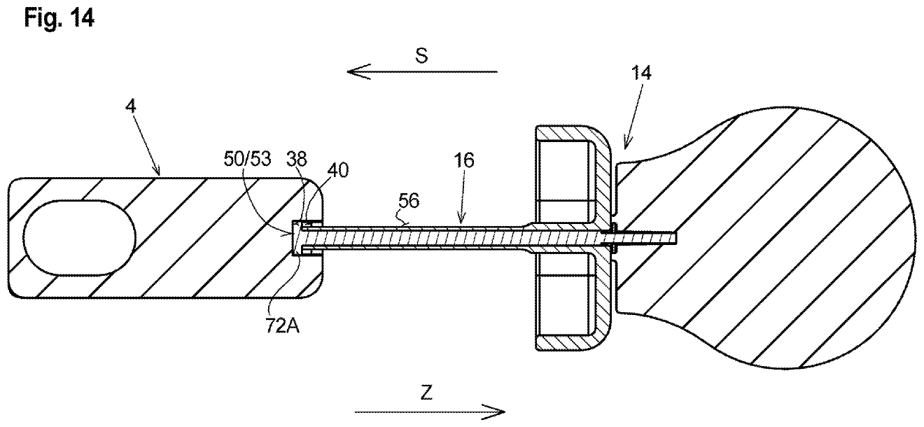

FIG. 14 a longitudinal section through the clamping element according to FIG. 11 in the attached state on an applicator,

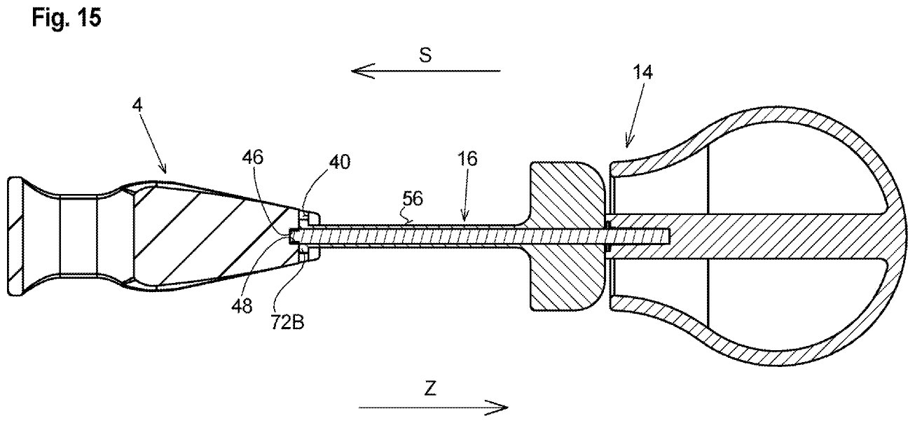

FIG. 15 a longitudinal section through the clamping element according to FIG. 11 in the attached state on an applicator, rotated by 90° compared to FIG. 14,

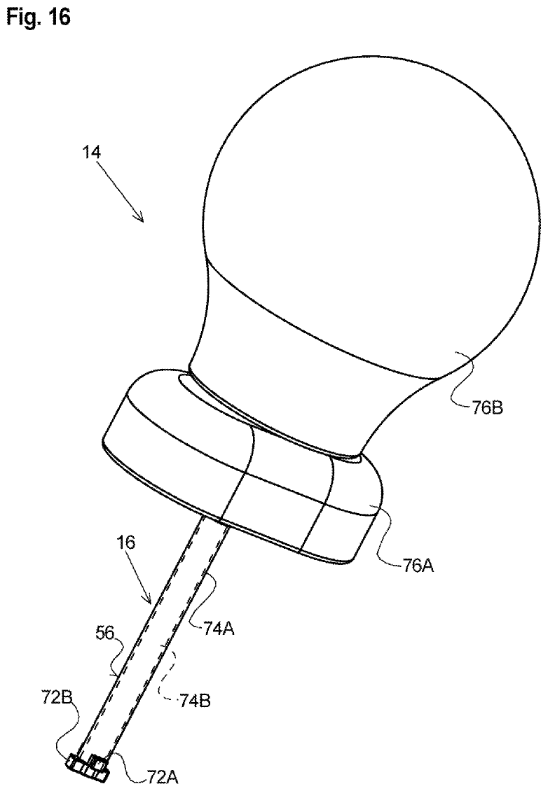

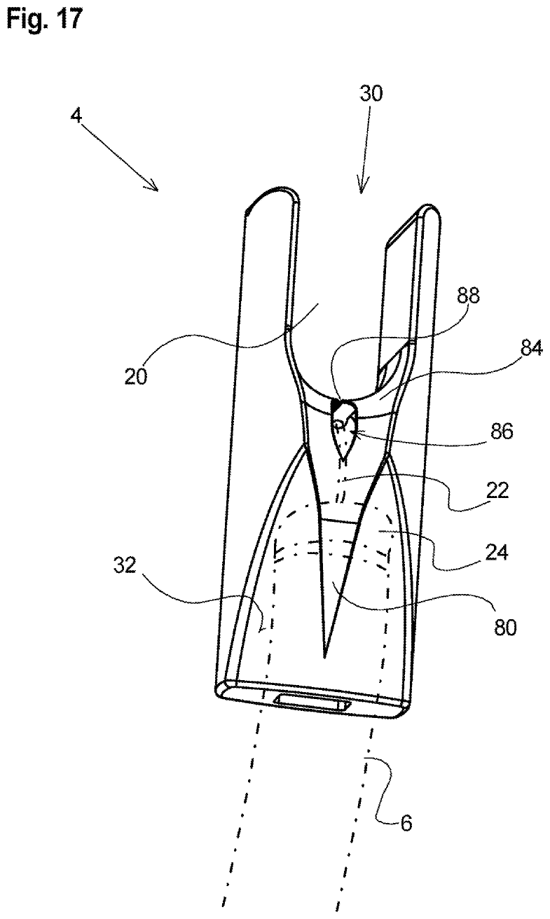

FIG. 16 a perspective view of the applicator shown in FIGS. 14 and 15 with the clamping element removed,

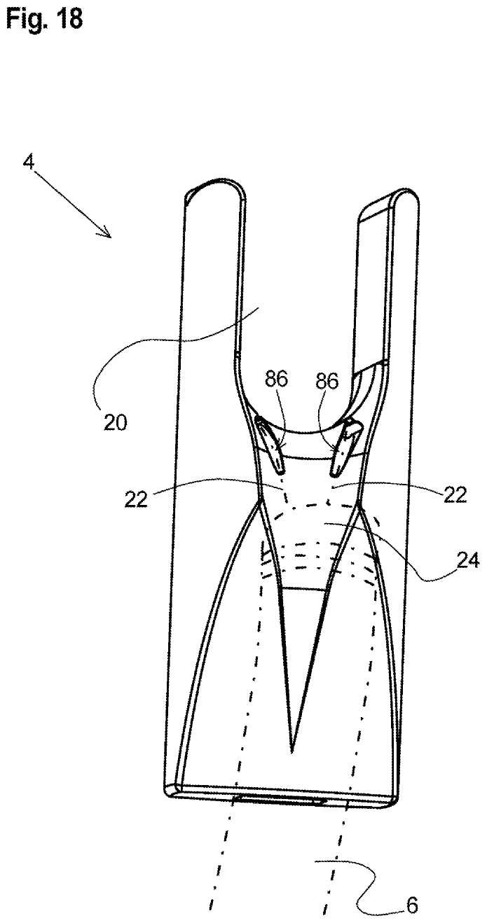

FIG. 17 a perspective view of a further alternative embodiment of the clamping element with an open recess, and

FIG. 18 a perspective view of a further alternative embodiment of the clamping element with a double thread guide.

FIG. 1 shows a setting assembly 2 with a clamping element 4 for the clamped fixation of a tissue strand 6, such as a tendon, a tendon substitute, or a band, such as a cruciate band substitute, to a securing recess 10 let into a bone 8. For this purpose, the securing recess 10 is formed by a bore which is additionally undercut in such a way that it tapers, at least in sections, in the direction of an entry opening 12.

In addition, the setting assembly 2 comprises an applicator 14 having an applicator shaft 16 to the distal end 18 of which the clamping element 4 can be releasably attached. The applicator 14 serves to insert the clamping element 4 into the securing recess 10 and to bring it into a suitable position there or to hold it in this position. For this purpose, a guide thread 22 already threaded in advance at a recess 20 of the clamping element 4 is carried along, which is connected to the tissue strand 6 to be fastened. By pulling on the guide thread 22, the tissue strand 6 is then guided within the securing recess 10 through the recess 20 and deflected at the latter, as shown by dash-dotted lines. Once one end 24 of the tissue strand 6 has re-emerged from the securing recess 10 of the bone 8, the tissue strand 6 deflected at the clamping element 4 can be clamped in the securing recess 10 by appropriately displacing the clamping element 4 in the direction of the entry opening 12 by means of the applicator 14. Should the deflected tissue strand 6 assume a clamping position which does not correspond to the desired position, the clamping element 4 can be released from the clamping position again by applying thrust by means of the applicator 14 and displaced into the securing recess 10 in order to subsequently clamp the deflected tissue strand 6 in a corrected position in the securing recess 10. During this entire process, the tissue strand 6 can thereby be subjected to a tensile force K via the guide thread 22 in order to fix it to the securing recess 10 with a desired pretension. In this clamping position and with the pretension applied, the tissue strand can then grow together with the bone 8, as in particular in the region of the cortex.

As can be seen from FIG. 2, the clamping element 4 has a clamping section 26 which tapers along a central axis A towards a first end 28 of the clamping element 4. The recess 20 at which the tissue strand 6 can be received and deflected is arranged at a second end 30 of the clamping element 4 facing away from the first end 28, so that after threading the tissue strand 6 extends along two opposite sides of the clamping section 26 at which the clamping element 4 forms lateral abutment surfaces 32 extending between the recess 20 and the first end 28.

As can be seen from FIG. 2, roughenings 34 in the form of tooth structures can be formed on the lateral contact surfaces 32, which serve to increase the friction with respect to an adjacent tissue strand 6. Preferably, the roughenings 34 are inclined in the direction of a threading direction E of the tissue strand 6, in order to be able to counteract a backward displacement of the tissue strand 6.

As can further be seen from the embodiment of the clamping element 4 according to FIGS. 3 to 5, the clamping element 4 has a tool receptacle 36 at the first end 28, via which the clamping element 4 can be releasably attached to the distal end 18 of the applicator 14. As can be seen from FIG. 4, this forms first contact means 38 in the form of a receiving base via which the clamping element 4 can be subjected to a pushing force in a pushing direction S by means of the applicator 14 in order to position the clamping element 4 within the securing recess 10.

As can also be seen from FIG. 4, the tool receptacle 36 also has second contact means 40 which are formed by at least one locating region 44 which is formed, by way of example, in the form of a rear gripping recess let into the side wall 42. In this case, the second contact means 40 serve to produce a positive-locking, quasi-positive or purely non-positive connection with the applicator 14, which connection is effective in a tensile direction opposite to the direction of thrust S, in order to be able to apply a tensile force Z to the clamping element 4.

In this embodiment according to FIG. 5, the tool receptacle 36 is formed by a polygonal holder with an exemplary rectangular cross-section. The rectangularly circumferential side wall 42 of the polygonal receptacle thus forms first rotation-locking means 46 in the form of rotation stops, by means of which a transmission of first torques M1 from the applicator 14, effective around the center axis A in a first direction of rotation D1, can be effected, and second rotation-locking means 48 in the form of rotation stops, by means of which a transmission of second torques M2 from the applicator 14, effective around the center axis A in a second direction of rotation D2, can be effected.

As can be seen from the setting assembly 2 according to FIG. 6 with the clamping element 4 according to FIGS. 3 to 5, the applicator shaft 16 forms an insertion end 52 at the distal end 18, to which the clamping element 4 can be attached. In this regard, an end face 53 of the insertion end 52 forms first mating contact means 50 which can be applied to the first contact means 38 when the clamping element 4 is attached, in order to enable a force transmission in the thrust direction S. In addition, second counter-contact means 54 are provided at the insertion end 52, which cooperate with the second contact means 40 in order to enable a force transmission in the pulling direction Z.

The insertion end 52 itself and/or the second counter-contact means 54 arranged on it also act as rotating means 55 of the applicator 14 which, in the connection position shown in FIG. 6, are simultaneously in direct contact with both rotation-locking means 46, 48 of the clamping element 4 and via which the torques M1, M2 can thus be generated.

The second counter-contact means 54 may thereby be formed by latching means which are displaced radially outwardly so as to project beyond a lateral surface 56 of the applicator shaft 16 and thereby, in the fitted state of the clamping element 4, engage in the second contact means 40 thereof or cooperate with the locating region 44. As exemplarily shown in FIG. 6, the counter-contact means 54 can be formed, for example, by radially displaceable latching bodies 58 which project beyond the lateral surface 56 in a latching position.

In this regard, as shown, the applicator 14 may include an adjusting pin 60 displaceable within the applicator shaft 16. In the position shown, this adjusting pin 60 blocks a radially inward displacement of the latching bodies 58. In this way, a positive engagement of the latching bodies 58 on the second contact means 40 of the clamping element 4 can be ensured. By actuation of, for example, a button-shaped handle 62 on an applicator handle 64, the adjusting pin 60 can be displaced distally from the illustrated latching position, counter to a spring F, into a release position. In this position, the latching bodies 58 can be displaced radially inwardly into pin recesses 61 of the adjusting pin 60 and thus into the lateral surface 56 (not shown). In this release position, the clamping body 4 can thus be released from the insertion end 52 of the applicator 14.

FIGS. 7 to 9 show another alternative embodiment of the clamping element 4, which has a tool receptacle 36 at the first end 28, in which the second contact means 40 are formed by a circumferential receiving groove 66.

Also, in this embodiment according to FIGS. 7 to 9, the tool receptacle 36, as can be seen in particular from FIG. 9, is formed by a polygonal holder which, however, has an exemplary hexagonal cross-section. Here, the hexagonal circumferential side wall 42 of the polygonal receptacle forms the first rotation-locking means 46 in the form of rotation stops, by means of which the first torques M1 can be transmitted in the first direction of rotation D1, and the second rotation-locking means 48 in the form of rotation stops, by means of which the second torques M2 can be transmitted in the second direction of rotation D2. For transmitting the torques M1, M2 from the applicator 14, its insertion end 52 has a polygonal profile adapted to the side wall 42. In the connection position shown in FIG. 10, the insertion end 52 thus forms the rotating means 55 bearing against the rotation-locking means 46, 48 in both directions of rotation D1, D2.

Moreover, as can be seen from the setting assembly according to FIG. 10 with the clamping element 4 according to FIGS. 7 to 9, the end face 53 of the insertion end 52 also forms the first mating contact means 50 in this embodiment, which can be applied to the first contact means 38 when the clamping element 4 is inserted, in order to allow force transmission in the direction of thrust S. The second counter-contact means 54, on the other hand, are formed by a spring washer 68 which is held on the lateral surface 56 and interacts with the second contact means 40 and the receiving groove 66, respectively, in order to permit a force transmission in the pulling direction Z in a force-fitting manner.

FIGS. 11 to 13 show a further alternative embodiment of the clamping element 4, which has a tool receptacle 36 at the first end 28, in which the first and second contact means 38, 40 are formed by a bayonet receptacle 70 for forming a bayonet connection with the applicator 14. In this regard, the bayonet receptacle 70 comprises a first bayonet receptacle 70A and a second bayonet receptacle 70B offset therefrom along the central axis A.

The first bayonet receptacle 70A thereby forms the first anti-rotating means 46 and the second rotation-locking means 48 in the form of rotation stops, via which the first torques M1 can be transmitted by the applicator 14 in the first direction of rotation D1 and the second torques M2 can be transmitted by the applicator 14 in the second direction of rotation D2. The second bayonet receptacle 70B forms the second contact means 40, for the application of force by the applicator 14, effective in the pulling direction Z.

As can be seen from the setting assembly according to FIG. 14 with the clamping element 4 according to FIGS. 11 to 13, the first mating contact means 50 are formed by a first bayonet cam 72A arranged at the distal end 18 of the applicator shaft 16 and cooperating with the first contact means 38 and the first bayonet receptacle 70A, respectively, in order to allow a force transmission in the pushing direction S by force.

In this respect, as can be seen from FIG. 15, the first bayonet receptacle 70A is slot-shaped and is adapted in shape and size to the bayonet cam 72A in such a way that, in the illustrated connecting position of the applicator 14 with the clamping element 4, it forms the first and second rotation-locking means 46, 48. In the connected position, the first bayonet cam 72A bears against these simultaneously in both directions of rotation D1 and D2 and in this way forms the rotating means 55. As a result of the simultaneous direct bearing, the first and second torques M1, M2 can thereby be transmitted directly from the applicator 14 to the clamping element 4 without any idle travel, even when the directions of rotation are changed.

In particular, as can be seen from FIG. 16, the applicator 14 shown in FIGS. 14 and 15 has a two-part applicator shaft 16. An outer first partial shaft 74A, which is connected to a first handle part 76A, thereby forms the lateral surface 56 with the first bayonet cam 72A projecting therefrom. The second bayonet cam 72B, on the other hand, is provided on a second partial shaft 74B arranged inside the first partial shaft 74A, which second partial shaft 74B is connected to a second grip part 76B and can be rotated relative to the first partial shaft 74B or its first bayonet cam 72A.

As a result, the second bayonet cam 72B can initially be inserted into the tool receptacle 36 of the clamping member 4 in alignment with the first bayonet cam 72A. Once the first bayonet cam 72A is received within the first bayonet receptacle 70A, the second bayonet cam 72B can be rotated relative thereto within the second bayonet receptacle 72B, such as by 90°, to thereby apply it in the direction of pull Z against the second contact means 40 formed by the second bayonet receptacle 70B, as can be seen in FIG. 15. In order to be able to fix an assumed angular position of the two bayonet cams 72A, 72B in relation to one another, they can be fixed to one another by means of latching or locking means provided, for example, between the handle parts 76A, 76B (not shown).

Due to the two bayonet cams 72A, 72B which can be rotated relative to each other, in the connected position with the clamping element 4 a direct transmission of force is thus possible both in the pushing direction S and the pulling direction Z and a transmission of the first and second torque M1, M2, respectively, is possible in both directions of rotation D1 and D2, without an idle travel without transmission of force or torque occurring during a change of direction or direction of rotation. In order to separate the applicator 14 from the clamping element 14, the two bayonet cams 72A, 72B must be rotated back into a common alignment in order to be able to be removed again from the tool receptacle 36.

In all embodiments according to FIGS. 1 to 15, the clamping element 4 has a recess 20 which is formed in the shape of a closed eyelet. In addition, the recess 20 has an elliptical shape in order to be able to thread the tissue strand 6 to be fixed more easily.

Alternatively, the recess 20 according to the embodiments shown in FIGS. 17 and 18 may be U-shaped and thereby open towards the second end 30 to allow even easier threading and deflection of the tissue strand 6 to be fixed.

In all embodiments, the clamping element 4 may further comprise guide grooves 80 recessed into the two lateral contact surfaces 32 and thereby directed towards the recess 20. When threading and deflecting the tissue strand 6, better centering relative to the recess 20 can be achieved by these guide grooves 80.

As can be seen in particular from FIGS. 4, 8 and 12, a groove bottom profile 82 of these guide grooves 80 thereby has different pitches in order to be able to adapt different sections of the clamping element 4 to their respective function during deflection and in particular during clamping of the tissue strand 6 deflected at the clamping element 4. Thus, the groove bottom profile 82 has a convex contour at the level of the clamping section 26 and the roughening 34 preferably formed therein (according to FIG. 2), in order to generate the greatest clamping forces in this region, at least in tendency, and thereby to achieve maximum stability of the clamped fixation.

In addition, in all embodiments of the clamping element 4, the guide groove 80 opens into the respective recess 20 via a rounded deflection region 84 in order to avoid, as far as possible, jamming of the tissue strand 6 when it is threaded into the recess.

As shown by way of example for the embodiment of the clamping element 4 according to FIG. 17, a thread guide 86 can additionally be let into the recess 20, in which the guide thread 22 is held and guided in particular during the insertion of the clamping element 4 into the securing recess 10. In this respect, the thread guide 86 has an exit gap 88 which has a gap width relative to the guide thread 22 which is narrow enough to accommodate the latter in a manner preventing loss, or to fix the clamping element 4 to the guide thread 22 before and during insertion into the securing recess 25. Moreover, the guide thread 22 can exit the thread guide 86 via this exit gap 88 as soon as the end 24 of the tissue strand 6 passes the recess 20.

In order to be able to guide the end 24 of the tissue strand 6 during deflection in a particularly stable manner and with a larger deflection radius in relation to the clamping element 4, a double thread guide 86 can also be provided on the recess 20, as shown in FIG. 18. This enables the use of two spaced-apart guide threads 22, both of which are attached to the end 24 of the tissue strand 6. These guide the tissue strand 6 into the securing recess 10, around the clamping element 4, through the recess 20 and back out of the entry opening 12. When tension is applied via the at least one guide thread 22, the tissue strand 6 can then be clamped in the securing recess 10 with the aid of the clamping element 4 guided on the applicator 14, so that it can later fuse with the bone 8 in the fixed position and, if necessary, under tension.

It is noted that all the elements and features of the various embodiments of the article according to the invention described above are interchangeable or combinable with one another, provided that an interchange or combination thereof is not precluded for technical reasons.

1. A setting assembly for the clamped fixation of a tissue strand to a fixing recess let into a bone, comprising:
a clamping element having a clamping portion tapering along a central axis of the clamping element towards a first end, a recess provided at a second end remote from the first end for the passage of the tissue strand, and a tool receptacle embedded at the first end which has and having a first contact configured to apply a force acting in a pushing direction and a second contact configured to apply a force acting in a pulling direction in the opposite direction to the pushing direction; and
an applicator having an insertion end for releasable connection to the tool receptacle, the applicator having a first mating contact configured to be applied to the first contact and a second mating contact configured to be applied to the second contact,
wherein the tool receptacle has a first rotation lock and a second rotation lock, and the insertion end of the applicator has a rotating device that can be applied in a first direction of rotation and in a second direction of rotation, opposite to the first direction of rotation, for transmitting first and second torques to the first and second rotation locks.

2. The setting assembly according to claim 1, wherein in a connection position both of the first and second contacts can be applied simultaneously to the first and second mating contacts, and the rotating device can be applied simultaneously to the first and second rotation locks.

3. The setting assembly according to claim 2, wherein in the connected position a form closure is formed both between the first and second contacts and first and second mating contacts, and between the rotating device and first and second rotation locks.

4. The setting assembly according to claim 1, wherein the first mating contact is formed by a distal shank end of an applicator shank and the second mating contact is formed by a latch, the latch being displaceable between a release position and a latching position and, in the latching position, projects beyond a peripheral surface of the applicator shank for forming a frictional or positive engagement with an undercut recess of the tool receptacle.

5. The setting assembly according to claim 4, wherein the latch comprises a spring washer.

6. The setting assembly according to claim 4, wherein the latch comprises latching bodies, the latching bodies being displaceable and fixable in the latching position.

7. The setting assembly as claimed in claim 6, wherein the applicator has an adjusting pin displaceable within the applicator shaft the adjusting pin blocking radial inward displacement of the latching bodies in a latching position and releases the latching bodies in a release position.

8. The setting assembly according to claim 1, wherein the first and second locks are formed by a polygonal receptacle acting in the first and second directions of rotation and the rotating device is formed by a polygonal profile provided at a distal end of the applicator shaft.

9. The setting assembly according to claim 1, wherein the rotating device comprises a bayonet cam engageable with a bayonet receiver of the tool receiver.

10. An applicator assembly according to claim 9, wherein the bayonet receptacle comprises a first bayonet receptacle and a second bayonet receptacle arranged offset thereto, and a first bayonet cam receivable in the first bayonet receptacle and a second bayonet cam receivable in the second bayonet receptacle are provided on the applicator, which is rotatably held with respect to the first bayonet cam.

11. The applicator assembly of claim 10, the first bayonet cam is connected to a first handle portion via a first partial shaft and the second bayonet cam is connected to a second handle portion of the applicator via a second partial shaft.

12. A clamping member for use in the setting assembly according to claim 1, wherein two lateral abutment surfaces are provided on opposite sides of the clamping portion and extending between the recess and the first end.

13. The clamping member according to claim 12, wherein the recess is formed in a closed eyelet shape.

14. The clamping member according to claim 12, wherein the recess is U-shaped and open towards the second end.

15. The clamping member according to claim 12, wherein a guide groove is let into at least one of the lateral contact surfaces in the direction of the recess.

16. The clamping member according to claim 15, wherein the guide groove comprises a groove bottom profile with different pitches.

17. The clamping member according to claim 16, wherein the groove bottom profile forms a convex contour in the clamping section.

18. The clamping member according to claim 15, wherein the guide groove opens into the recess via a rounded deflection region.

19. The clamping member according to claim 12, wherein an additional thread guide for a guide thread is let in at the recess.

20. The clamping member according to claim 19, wherein the additional thread guide comprises an exit gap.

21. The clamping member according to claim 19, wherein the additional thread guide is designed as a double thread guide for two guide threads guided at a distance from one another.

22. The clamping member according to claim 12, wherein roughenings are formed on the lateral contact surfaces for increased friction with respect to an abutting tissue strand.

23. The clamping member according to claim 22, wherein the roughenings are formed by tooth structures, teeth of the tooth structures being at least partially inclined in the direction of a threading direction on both lateral contact surfaces.

24. The clamping member according to claim 23, wherein the roughenings extend with respect to the central axis at a level of a convex portion of the groove bottom profile.

25. An applicator for use in the setting assembly according to claim 1.
